(19) United States
(12) Patent Application Publication
(10) Pub. No.: US 2019/0125995 A1
(43) Pub. Date: May 2, 2019

(54) Oral Airway

(71) Applicant: Alberto Otero, Lake Havasu City, AZ (US)

(72) Inventor: Alberto Otero, Lake Havasu City, AZ (US)

(21) Appl. No.: 15/795,269

(22) Filed: Oct. 27, 2017

(51) Int. Cl. A61M 16/04; A61M 16/08

(52) U.S. Cl. CPC A61M 16/0463; A61M 16/0816; A61M 16/0402; A61M 2210/065; A61M 2230/432; A61M 16/0488; A61M 2016/0413

(57) ABSTRACT

The invention provides an oral airway device that comprises three channels, which extend from the proximal to the distal end. The first channel is for supplying oxygen, the second is for the suctioning of fluids, and the third is for measuring carbon dioxide. The device allows for the patient's spontaneous respirations and provides other utility. The device is placed through the patient's mouth until the proximal end contacts the outside surface of the patient's lips, so that the distal end of the device reaches an area in the patient's throat above the trachea and the epiglottis. The device reduces the possibility for nasal mucosa inflammation, the risk of infection, and other medical complications. The device also allows the healthcare provider to insert it without first using a lubricant or spray. The device remains firmly in the correct position without the use of a strap.

### CROSS-REFERENCE TO RELATED APPLICATION

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR

Not Applicable

### BACKGROUND

Unless otherwise indicated herein, the materials described in this section are not prior art to the claims in this application and are not admitted to be prior art by inclusion in this section.

### 1. Field of the Invention

The present invention generally relates to medical devices. More specifically, the present invention relates to medical devices that allow for supplying oxygen to a patient.

### 2. Description of Related Art

The medical field is continuously progressing with the development of technology and research. Engineers and medical professionals work together to develop better tools that may be used in emergency situations. Modern medicine relies on specific equipment for specific procedures. For example, surgeons rely on various tools designed specifically to help them complete medical procedures faster and with minimal invasiveness to the patient. Furthermore, in life threating situations, having the correct device for the job can make the difference between life and death.

People often look at ways to improve medical technology. Trained medical professionals use various tools that allow them to complete lifesaving procedures. Monitoring and maintaining a patient's vital signs such as pulse rate, respiration rate, temperature, and blood pressure requires specific tools. If a person is unresponsive, their jaw muscles and tongue may relax and obstruct their airway, which may cause suffocation.

The upper respiratory track is critical to survival, since it allows humans to breath. Trauma and other conditions may hinder a person's ability to breath on their own. Patients who are under anesthesia for surgical procedures may have difficulty maintaining a constant respiratory rate. Airway management is a medical procedure conducted when a patient requires assistance in breathing. It is used to allow medical professionals to prevent obstructions in the airway and ensure ventilation with an open pathway between the patient's lungs and the atmosphere.

Various airway management procedures may be conducted based on the severity of the condition of the patient. While the most basic procedures do not require invasive maneuvers, more severe cases may require the use of specialized equipment or advanced techniques. These severe cases include emergency situations where patients are unconscious or otherwise unable to sustain breathing on their own. Due to traumatic events or other conditions, patients may not be able to sustain normal breathing for extended periods of time. For example, since some comatose patients are unable to breath on their own, assisted breathing may be required for a long period of time.

One known method of delivering oxygen to a patient includes the use of nasal prongs, which are placed in the patient's nose as the primary oxygen delivery system. However, delivering oxygen with nasal prongs may cause irrigation to the nasal mucosal lining. This may lead to swelling of a mucous membrane, which may block the passage of oxygen causing nasal mucosa inflammation. Once the nasal passages are blocked or restricted, the patient often resorts to mouth breathing. Nasal breathing is preferred over mouth breathing, because it has the advantage of warming and moisturizing the air.

Another known method of delivering oxygen to a patient includes using an endotracheal tube, which is a flexible plastic tube used to open a patient's airway and prevent suffocation. Assisted breathing with an endotracheal tube can maintain the vital respiratory rate, for an extended period of time. The method of using an endotracheal tube to provide airway management is known as endotracheal intubation. When the endotracheal tube is inserted through a patient's mouth, it is known as an oral endotracheal tube. When it is inserted through a patient's nasal canal, it is known as a nasal endotracheal tube.

The endotracheal tube travels into the patient's trachea, which is commonly referred to as the windpipe. To insert the endotracheal tube into the trachea, it needs to pass through the epiglottis, which is the opening on the top of the trachea. The epiglottis is open when we breathe, but closes when we swallow to divert food and beverages to the esophagus and to prevent them from entering the trachea. An endotracheal tube forces the epiglottis into an open position. To reduce the amount of secretions that can travel down the outside of the endotracheal tube and into the lungs, the endotracheal tube usually contains a soft balloon around its outside diameter, which is commonly referred to as a cuff. The cuff is inflated with air to reduce secretions into the lungs. However, the cuff may not completely prevent these secretions. Thus, prolonged use of endotracheal tubes may have many adverse effects on the patient, such as increasing the risk of infection and causing nasal inflammation.

Other disadvantages with using an endotracheal tube is that it may need to be lubricated before it is inserted into the patient's mouth. It may requires a strap to be installed around the patient's head to keep it in place. Further disadvantages with using an endotracheal tube is that long duration of treatment may increase the risk of nosocomial pneumonia, because: (1) an endotracheal tube may injure the mucous membrane, which may reduce the effectiveness of coughing, (2) an injury to the mucous membrane may create a site for bacteria to bind, (3) endotracheal intubation may cause airway colonization, and (4) an endotracheal tube may serve as a reservoir for bacteria to remain sequestered, safe from host defenses, and enhance bacterial entry into the lungs. When microorganisms adhere to the surface of an endotracheal tube, some species may form a bacterial biofilm on the surface lining, which is similar to a slime-like adhesive. The bacterial biofilm is a complex matrix that allow nutrients to circulate analogous to tissues of higher organisms. The sessile forms of bacterial biofilm can give rise to planktonic bacteria that may eventually leave the biofilm and disperse into the environment.

Another known method of delivering oxygen to a patient includes using a laryngeal mask airway (LMA). An LMA is shaped like a large endotracheal tube on the proximal end, while the distal end comprises an elliptical-shaped mask with an inflatable cuff. The cuff allows air to be delivered down through the trachea and into the lungs, while blocking the esophagus to prevent air from entering into the stomach. Although the LMA is less invasive than an endotracheal tube, it also has its disadvantages. For example, the LMA needs to be lubricated. It is also possible to either underinflate or overinflate the cuff on the LMA. If the cuff is underinflated, it can result in a poor mask seal, which could potentially cause the stomach to swell from air pressure, pushing up the diaphragm, limiting lung volume, and increasing the risk of aspiration. If the cuff is overinflated, the oropharyngeal mucosa can be easily damaged resulting in a sore throat, nerve injury, and discomfort while swallowing. Also, the LMA may cause partial airway obstruction if the mask is not correctly placed. In addition, the LMA must be connected to a conventional anesthesia circuit. Furthermore, the LMA does not provide a mechanism for suctioning secretions. Moreover, the LMA usually requires a strap to keep it in place.

As a result, there is a need for a device that (1) does not need to be lubricated, (2) can easily be kept in place without a strap, (3) does not injure the mucous membrane, (4) resists the accumulation of bacteria and bacterial biofilm, (5) provides a method for suctioning secretions, (6) reduces the risk of infection and pneumonia, and (7) avoids other medical complications.

### BRIEF SUMMARY OF THE INVENTION

It is a principal object to solve at least one of the disadvantages with other attempted solutions or to create other utility by providing an improved device for conducting airway management in patients that require assistance in sustaining breathing or otherwise improves upon the prior art that (1) does not need to be lubricated, (2) can easily be kept in place without a strap, (3) does not injure the mucous membrane, (4) resists the accumulation of bacteria and bacterial biofilm, (5) provides a method for suctioning secretions, (6) reduces the risk of infection and pneumonia, (7) avoids other medical complications, or (8) provides other utility.

The present invention provides an oral airway device. The device comprises three channels that extend from the proximal end to the distal end. The first channel is for supplying oxygen, the second is for the suctioning of fluids, and the third is for measuring carbon dioxide in the air that the patient exhales. The device allows for the patient's spontaneous respirations and provides other utility. The distal end of the device is placed through the patient's mouth until the proximal end contacts the outside surface of the patient's lips. In this way, the distal end of the device reaches an area in the patient's throat above the trachea and the epiglottis. This placement avoids complications that are associated with delivering oxygen to the patient by other methods. The device reduces the possibility for nasal mucosa inflammation, the risk of infection, and other medical complications. The device also allows the healthcare provider to insert it without first using a lubricant or spray. The device remains firmly in the correct position without the use of a strap. This allows medical personnel to retain the use of their hands to perform other tasks during surgical procedures.

### BRIEF DESCRIPTION OF THE DRAWINGS

The accompanying drawings, which are incorporated in and constitute a part of this specification, illustrate several aspects described below.

FIG. 1 is a top perspective illustration of the present invention in which at least one of the embodiments of this invention is implemented.

FIG. 2 is a side perspective illustration of the present invention, with various attachments connected in which at least one of the embodiments of this invention is implemented.

FIG. 3 is another side perspective illustration of the present invention, with various attachments connected in which at least one of the embodiments of this invention is implemented.

### DETAILED DESCRIPTION OF THE INVENTION

It is to be understood that this invention is not limited to any particular embodiment described, which may vary. Also, it is to be understood that the terminology used herein is for the purpose of describing particular embodiments only, and is not intended to be limiting, since the scope of this invention will be limited only by the appended claims.

In the following detailed description, numerous specific details are set forth in order to explain and provide a thorough understanding of the present invention. However, it is apparent that the present invention may be practiced without all of these specific details. Thus, all illustrations of the drawings are for the purpose of describing versions of the present invention, and are not intended to limit the scope of the invention.

In the following section, the present invention is described fully by referencing the details in the enclosed drawings, which illustrate certain embodiments of the invention. The numbers shown in this specification refer to the corresponding numbers in the enclosed drawings. The terminology used is to describe the particular embodiment shown and is not intended to limit the scope of the invention. The invention may also be embodied in many other forms in addition to the embodiments shown. Thus, the embodiments shown should not be construed as limiting, but rather, to allow a thorough and complete description of the disclosure that conveys the scope of the invention to a person having ordinary skill in the art in the field of this invention. Therefore, for the terms used herein, the singular forms “the,” “a,” and “an” are intended to include the plural forms as well as the singular forms, unless the context clearly indicates otherwise. The term “and” includes any and all combinations of one or more of the associated listed items. As used herein, the terms “comprising” and “comprises” when used in this specification, identify specific steps, integers, operations, features, components, and elements, but do not preclude the presence or addition of one or more other steps, operations, features, components, and elements. In addition, the features, components, and elements referenced may be exaggerated for clarity.

Unless otherwise defined, all scientific terms, technical terms, or other terms used herein have the same meaning as the term that is understood by one having ordinary skill in the art in the field of this invention. It is also understood that these terms, including their dictionary meaning, should be understood as having the meaning, which is consistent with their definitions in the related relevant art. In addition, the present disclosure is not to be interpreted in an idealized or overly formal sense unless expressly stated so herein. Constructions or functions that are well known in the art may not be fully described in detail for brevity.

In describing the invention, it is understood that a number of steps and methods may be disclosed. Each of these may have individual benefit. Also, each may be used in conjunction with at least one or more of the disclosed steps and methods. Therefore, this description will refrain from stating each and every possible combination of the individual steps and methods for the sake of brevity. Regardless, the specification and related claims should be understood with the combinations that are entirely within the scope of the claims and inventions.

The disclosure in this invention are examples of how it may be implemented and are not intended to limit the scope of the invention to the specific embodiments shown in the accompanying drawings or the description provided herein. All illustrations are for the purpose of describing selected versions of the present invention and are not intended to limit the scope of the present invention. The present invention will now be described by example in the following paragraphs by referencing the accompanying drawings, which represent embodiments and alternative embodiments. All illustrations of the drawings are for the purpose of describing selected versions of the present invention and are not intended to limit the scope of the present invention.

In the prior art, when nasal prongs are used as the primary oxygen delivery system, nasal mucosa inflammation occurs, which leads to undesirable mouth breathing. The unique design of the present invention allows oxygen to be supplied closer to the trachea, which reduces the possibility for nasal mucosa inflammation. Supplying oxygen closer to the trachea also allows for the suctioning of unwanted secretions, which allows for an uninterrupted supply of oxygen. In addition, the device of the present invention allows a healthcare provider to retain the use of her hands, which allows her to conduct other tasks. In at least one embodiment, the present invention can combine an oxygen to carbon dioxide splitter with extensions. The oral airway device described in this invention is more effective in supplying oxygen to a compromised patient with a. blocked airway than other methods, because the oral airway device delivers oxygen above and in close proximity to a patient's trachea.

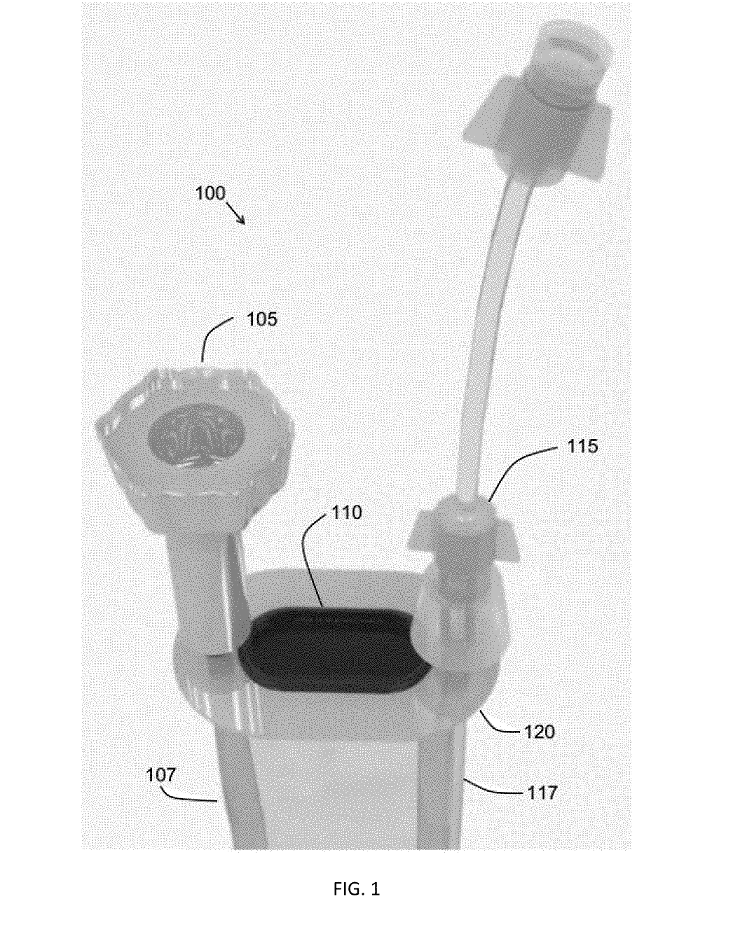

In reference to FIG. 1, the present invention is an oral airway device 100 designed to maintain an open airway when a person is unconscious. The device 100 has a unique design to allow for supplying oxygen to an area above the epiglottis and trachea, which is in close proximity to the trachea. FIG. 1 shows the device 100 with a larger oxygen (O2) connector 105 on the left, a smaller carbon dioxide (CO2) connector 115 on the right, and a center suctioning channel 110.

FIG. 1 also shows an O2 channel 107, which extends from the proximal to the distal end of the device 100. The O2 connector 105 connects to the O2 channel 107 to allow oxygen to be supplied from the proximal end to the distal end of the device 100.

Similarly, a CO2 channel 117 extends from the proximal end to the distal end of the device 100. The CO2 connector 115 connects to the CO2 channel 117 to allow carbon dioxide to travel from the distal end to the proximal end of the device 100.

In a similar manner, the center suctioning channel 110 extends the entire length of the oral airway from the proximal end to the distal end of the device 100. This allows for fluids to be suctioned from the distal to the proximal end of the device 100.

The pigtail for the CO2 channel 115 must extend at least 3 mm above the opening for channel 110. The CO2 exhaled by the patient's spontaneous respirations while using the oral airway device 100 described in this invention will be monitored by the CO2 channel 115, which is connected to a capnography that measures how much carbon dioxide is present in the patient's breath.

As shown in FIG. 1, the device 100 comprises a large channel 110 that may be used to suction fluids, such as emesis, vomit, stomach contact, mucus, and blood, which may either accumulate or be inhaled into the patient lungs. This large channel 110 may also be used when secretions accumulate in the oropharynx. When the oral airway is being used in its normal function to assist the patient in breathing, vacuuming through the large channel 110 may not be required. Also, as shown in FIG. 1, a large flair 120 is connected to the proximal end of the device. This flair 120 rests against the patient's lips to prevent him from swallowing the oral airway device 100.

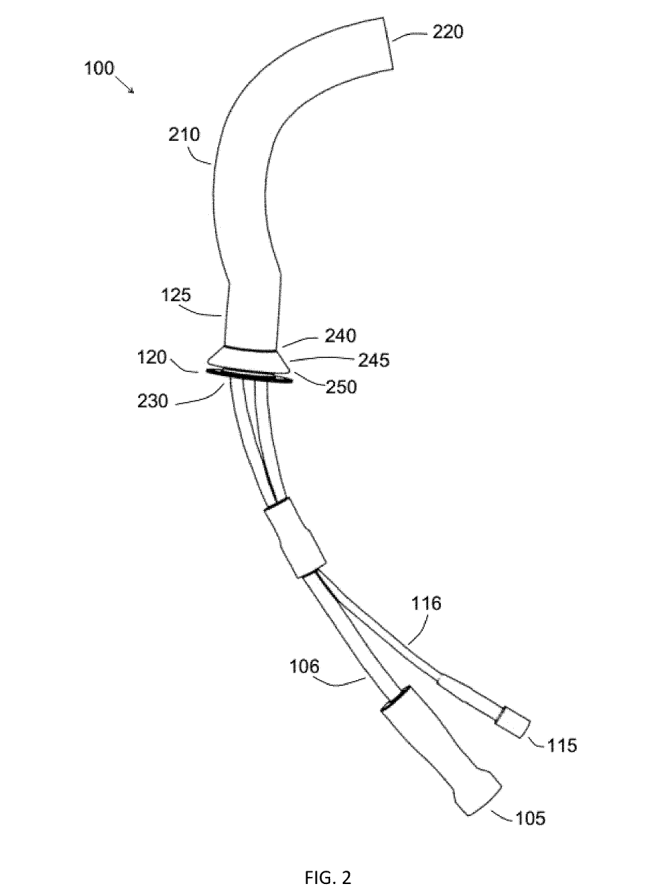

In reference to FIG. 2, the device 100 comprises a plurality of universal oxygen connectors posited at the proximal end 230, which are able to connect to standardized equipment used in medical facilities. FIG. 2 also shows the larger O2 channel on the left 105, and the smaller CO2 channel on the right 115. The versatility of the device allows medical professionals to integrate the device 100 in the equipment that the majority of the medical community uses today.

As shown in FIG. 2, the oral airway device 100 is unique by having an oxygen connector 105 with an oxygen supply connection at the proximal end 230 and an opening at the distal end 220. An oxygen supply system may connect to the oxygen tubing with the oxygen connector 105 near the proximal end of the device 230. Oxygen then flows through the oxygen connector 105 to the distal end of device 220. An oxygen supply regulator may be used to force oxygen thru the device 100 to the proximal end 230. In at least one embodiment of the invention, the oxygen tubing 106 may extend from the oxygen connector 105 to the proximal end 230 of the device 100. Then from the proximal end 230 of the device 100, the oxygen may travel through the oxygen channel 107 without the use of tubing to the distal end 220 of the device 100. In at least one other embodiment of the invention, the oxygen tubing 106 may extend from the oxygen connector 105 through the oxygen channel 107 to the distal end 220 of the device 100, where the oxygen tubing 106 runs through the oxygen channel 107.

When the oral airway device 100 is inserted into a patient's mouth, the distal end 220 extends inside the patient until the distal end 220 reaches a point that is above the epiglottis. The epiglottis is the flap of cartilage at the root of the tongue that is depressed during swallowing to cover the opening to the windpipe. Thus, the distal end 220 of the oral airway device 100 is also above the trachea, which is commonly referred to as the wind pipe. This configuration allows the device 100 to maintain a patient's airway and deliver oxygen to the lungs through the trachea.

The distal end 220 of the oral airway device 100 is primarily placed in the oropharynx, which is the area of the pharynx that can be seen when you look into the mouth towards the throat. The oropharynx lies above the laryngopharynx, also known as the hypopharynx, which is located adjacent to the epiglottis and above the esophagus. The oropharynx also lies below the nasopharynx, which is the upper part of the throat that lies behind the nose, above the soft part of the roof of the mouth (soft palate), and just in back of the nasal passages.

Since the distal end 220 of the oral airway device 100 lies above the epiglottis, inserting it into a patient's mouth in the region of the oropharynx does not require endotracheal intubation. Thus, the oral airway device 100 does not require an endotracheal tube to pass through the epiglottis. Unlike an endotracheal tube, which forces the epiglottis into an open position, the oral airway device 100 does not interfere with the operation of the epiglottis. Also, the oral airway device 100 does not require a cuff, which is the inflated soft balloon that is placed around the outside diameter of the endotracheal tube to reduce the amount of secretions that can travel down the outside of the endotracheal tube and into the lungs. Since no cuff is required, the risk of secretions into the lungs, which can cause pneumonia, is eliminated through the use of the oral airway device 100.

The oral airway device 100 may be used to deliver oxygen to an unresponsive patient with a blocked airway by providing an open and unobstructed airway without requiring endotracheal intubation, a laryngeal mask, a cuffed airway, or a strap, while firmly staying in place. Also, FIG. 2 shows the location of a straight section of the device known as the bite block 125, which prevents the patient from biting through the device. In at least one other embodiment of this invention, the straight cylindrical portion 125 of the oral airway device 100 may have a band of soft flexible material around the bite block 125 to allow the patient to bite into. This allows the patient to relax his jaw and allow his teeth to sink into the soft material, which may also assist in keeping the oral airway device 100 in position in relation to its proximity to the trachea.

In addition, as shown in FIG. 2, in at least one other embodiment of the invention, the device 100 may comprise a converging section 245 around the outside circumference of the bite block 125. The converging section 245 has its smallest diameter at the distal end 240, which is approximately the same as the diameter of the bite block 125. The converging section 245 has its largest diameter at the proximal end 250, which is approximately the same as the diameter of the flair 120. The converging section 245 is installed so that a small gap remains between its proximal end 250 and the flair 120. This gap should be slightly wider than the width of the patient's front teeth, so that a patient may snugly insert his front teeth between the proximal end 250 and the flair 120. To do this, a healthcare worker would hold the device 100 near its proximal end 230, and insert the distal end 220 of the device 100 into the patient's mouth. In doing so, the ends of the front teeth will first slidingly glide along the outside diameter of the hollow curved tubular member 210 and then slidingly glide across the straight section of bite block 125. Next, the patient's front teeth slidingly glide from the distal end 240 of the converging section 245 to the proximal end 250. As the device 100 is pushed slightly further into the patient's mouth, the teeth will slide off the proximal end 250 and allow the patient to clamp down so that his front teeth are slidingly wedged between the proximal end 250 and the flair 120. In this configuration, the front of the patient's front teeth may touch or be in close proximity to the flair 120, and the back of the patient's front teeth may touch or be in dose proximity to the proximal end 250. In this configuration, the distance between the ends of the patient's front teeth and bottom teeth is approximately the same as the diameter of the bite block 125. Thus, the patient's front teeth remain in the gap between the flair 120 and the proximal end 250, which is the largest diameter of the converging section 245. This gap defines a retention compartment for preventing movement of the device 100 in relation to the patient's teeth without the use of a strap.

In at least one alternative embodiment, the position of converging section 245 may be adjusted by slidingly moving it along the straight cylindrical portion 125 of the oral airway device 100 towards the distal end 220 of the oral airway device 100. This allows the gap to be expanded between the proximal end 250 of the converging section 245 and the large flair 120, which is located near the proximal end 230 of the oral airway device 100. Expanding this gap may make it easier to accommodate a patient with thicker front teeth or a patient wearing jewelry, orthodontics, braces, or similar devices on their front teeth.

In at least one alternative embodiment, the position of the large flair 120 may also be adjusted by slidingly moving it along the straight cylindrical portion 125 of the oral airway device 100 towards the distal end 220 of the oral airway device 100. This allows the gap to be reduced between the large flair 120 and the proximal end 250 of the converging section 245. Reducing this gap may make it easier to accommodate a patient with thinner front teeth.

In at least one alternative embodiment, both the position of the large flair 120 and the converging section 245 may be adjusted along the straight cylindrical portion 125 towards the distal end 220 of the device 100. This will allow the distance between the distal end 220 of the device 100 and the patient's trachea to be adjusted. Thus, medical personnel may wish to adjust the position of both the large flair 120 and the converging section 245 until the desired distance is reached between the distal end 220 of the device 100 and the patient's trachea.

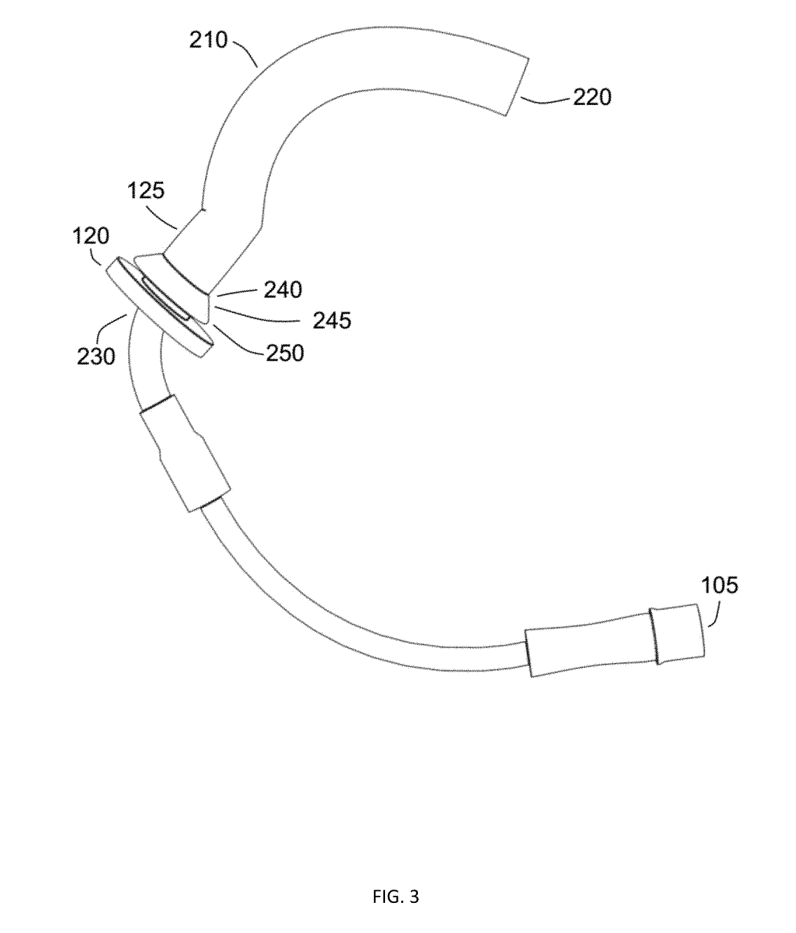

As shown in FIG. 3, in at least one embodiment of this invention, the large flair 120 at the proximal end 230 of the oral airway device 100 rests on the front teeth of the patient near his lips. The oral airway device 100 may be manufactured with various diameters to accommodate the various sizes of the mouths of patients. For example, a diameter of 60 mm may be used for a child, while a diameter of 90 mm may be used for an adult, and a diameter of 110 mm may be used for a larger adult. In addition, in at least one embodiment of this invention, the oral airway may also be manufactured in other diameters ranging from approximately 40 mm to 110 mm. These oral airway devices 100 may comprise diameters of 40 mm, 50 mm, 60 mm, 70 mm, 80 mm, 90 mm, 100 mm, and 110 mm. The oral airway devices 100 may also be color coded based on their diameters, so that, medical personnel may quickly select the correct sized oral airway device 100 based on the size of the patient and the color of the oral airway device 100.

Also as shown in FIG. 3, in at least one embodiment of this invention, beyond the proximal end 230 of the oral airway device 100, there may be a straight cylindrical portion 125 of the oral airway device 100. The straight cylindrical portion 125 of the oral airway device 100 may be reinforced to prevent the patient from biting into or through the oral airway device 100,

in reference to FIG. 3, an oral airway device 100 is illustrated for insertion into the mouth of the patient to provide a breathing pathway. The oral airway 100 has a hollow curved tubular member 210 with a proximal end 230 and a distal end 220. The distal end 220 is inserted into the mouth of the patient until it reaches an area above the epiglottis and above the trachea. In at least one embodiment of the present invention, the oral airway device 100 may be manufactured from a plastic non-toxic material, such as polyvinyl chloride, polypropylene, polyethylene, or polyethylene terephthalate (“PET”). In at least one alternative embodiment, the oxygen may flow directly from the oxygen connector 105 and through the oxygen tubing 106 to the oxygen channel 107, which is formed within the device 100, to the distal end 220 without the oxygen tubing 106 running through the oxygen channel 107 or within the device 100. In such a configuration, the device 100 may be constructed from a medical quality plastic such as PET, which is used to prevent oxygen permeability, so that the tubing can be connected only at the proximal end 230 of the device 100 without inserting tubing through the device 100. The force of the setting of the oxygen supply regulator will force the oxygen thru the device 100 where is intended.

FIG. 3 also shows the flair 120 at the proximal end 230 of the oral airway device 100, which forms a mouth guard to prevent the patient from swallowing the device 100 or from it being inserted to deeply into the patient's throat.

The oxygen supply connection 105 is located at the proximal end 230 near the lips of the patient. Oxygen tubing 106 may be used to connect the oral airway device 100 to the oxygen supply connection 105, which may connect to an oxygen source, such as a wall oxygen supply source or an oxygen tank with an oxygen regulator. The oxygen supply connection 105 that connects to oxygen tubing 106 may either terminate at the proximal end 230 of the oral airway device 100, or travel all the way through the oxygen channel 107 through the device to its distal end 220. In at least one embodiment, the oxygen may flow through oxygen tubing that is placed from the proximal end 230 to the distal end 220 of the device 100 within an oxygen channel 107 within the device 100.

in at least one other embodiment of this invention, right beyond the straight cylindrical portion 125 of the oral airway device 100 there is a curved portion 210 of the oral airway device 100 to allow for depression of the tongue. In at least one other embodiment of this invention, the distal end 220 of the oral airway device 100, may have a smooth tapered edge 220 to reduce or eliminate oral mucosal trauma when the oral airway device 100 is inserted into the patient.

Unlike other inventions, lubricant is not needed to insert the oral airway into a patient's mouth. Also, a strap is not needed to hold the device 100 in place like other devices. This is because the patient's front teeth hold the device 100 in position by snugly fitting in the gap or retention compartment between the flair 120 and the largest diameter of the section 245.

Overall, the device 100 of this invention may: (1) provide oxygen while monitoring the CO2 level of the patient having surgery both in a hospital or outpatient center, (2) provide oxygen and CO2 monitoring of the patient in a Coronary Care Unit (CCU), an Intensive Care Unit (ICU), a Post Anesthesia Care Unit (PACU), etc., and (3) allow for transportation of the patient needing oxygen from the field to a medical facility with little training to the medical personnel.

Although the invention has been explained in relation to its at least one embodiment, it is to be understood that many other possible modifications and variations can be made without departing from the spirit and scope of the invention as hereinafter claimed. All of these embodiments and the invention disclosed herein are intended to be within the scope herein disclosed. These and other embodiments of the invention will become readily apparent to those skilled in the art from the detailed description of the embodiments having reference to the attached figures, the embodiments not being limited to any particular embodiments disclosed. Also, the invention disclosed herein suitably may be practiced in the absence of any element which is not specifically disclosed herein.

What is claimed is:

1. An oral airway device for delivering oxygen to the vicinity of the trachea, comprising at least three channels within the device having a proximal end and a distal end.

2. The oral airway device according to claim 1 wherein the device comprises a flair at the proximal end of the oral airway device to prevent the patient from swallowing it.

3. The oral airway device according to claim 2 wherein the device comprises a connection at the proximal end of the oral airway device to allow for suction from the distal end of the device to the proximal end of the device.

4. The oral airway device according to claim 3 wherein the device comprises a connection at the proximal end of the oral airway device to connect to oxygen tubing to allow for delivery of oxygen from the proximal end of the device to the distal end of the device.

5. The oral airway device according to claim 4 wherein the device comprises a connection at the proximal end of the oral airway device for measuring the percentage of carbon dioxide in the air that the patient exhales from the distal end of the device to the proximal end of the device.

6. The oral airway device according to claim 5 wherein the device comprises a unique design at the distal end of the device so that when it is inserted in the patient's mouth, it rests above the epiglottis and above the trachea to allow for a close delivery of oxygen to the trachea.

7. The oral airway device according to claim 6 wherein the oral airway does not require endotracheal intubation, a laryngeal mask, or a strap.

8. The oral airway device according to claim 7 wherein the gas being supplied is oxygen.

9. The oral airway device according to claim 8 wherein the gas being monitored is carbon dioxide.

10. The oral airway device according to claim 9 wherein the diameter of the device is selected from the group consisting of 40 mm, 50 mm, 60 mm. 70 mm, 80 mm, 90 mm. 100 mm, and 110 mm.

11. The oral airway device according to claim 10 wherein the device is color coded based on its diameter.

12. The oral airway device according to claim 11 wherein the device may be inserted without the use of a lubricant or anesthesia.

13. The oral airway device according to claim 12 wherein the device may be made from a plastic non-toxic material selected from the group consisting of polyvinyl chloride, polypropylene, polyethylene, and polyethylene terephthalate.

14. The oral airway device according to claim 13 wherein the device comprises a curved portion to allow for depression of the tongue.

15. The oral airway device according to claim 14 wherein the device comprises a straight section referred to as a bite block to prevent a patient from biting through the device.

16. The oral airway device according to claim 15 wherein the device comprises a converging section on the outside circumference of the bite block to allow the teeth to glide across this section and remain in the area between the flair and the converging section defining a retention compartment for preventing movement of the device in relation to the patient's front teeth.

17. The oral airway device according to claim 16 wherein the position of the converging section may be adjusted by slidingly moving it along the bite block to allow the gap to be adjusted between the converging section and the flair.

18. The oral airway device according to claim 17 wherein the position of the flair may be adjusted by slidingly moving it along the bite block to allow the gap to be adjusted between the converging section and the flair. 19, The oral airway device according to claim 18 wherein both the position of the flair and the converging section may be adjusted by slidingly moving it along the bite block to adjust the distance between the distal end of the oral airway device and the patient's trachea.

20. A method for inserting an oral airway device into the mouth of a patient comprising the steps of:
a. adjusting the position of the converging section by slidingly moving it along the bite block until the desired distance between the distal end of the oral airway device and the patient's trachea is reached;
b. adjusting the position of the flair by slidingly moving it along the bite block until the desired distance between the flair and the converging section is reached;
c. inserting the oral airway device according to claim 19 into the mouth of the patient until the front teeth slidingly glide along the outside diameter of the hollow curved tubular member and the straight section of the bite block;
d. further pushing the oral airway device into the mouth of the patient until the front teeth of the patient slidingly glide from the distal end of the converging section towards the proximal end of the converging section; and
e. cease pushing the oral airway device into the mouth of the patient when the teeth slide off the converging section allowing the patient to clamp down so that the front teeth are wedged between the converging section and the flair, wherein this gap defines a retention compartment for preventing movement of the oral airway device in relation to the patient's teeth without the use of a strap.
